(19) United States
(12) Patent Application Publication
(10) Pub. No.: US 2013/0324842 A1
(43) Pub. Date: Dec. 5, 2013

(54) Method for Estimating Pressure Gradients and Fractional Flow Reserve from Computed Tomography Angiography: Transluminal Attenuation Flow Encoding

(71) Applicant: The Johns Hopkins University, Baltimore, MD (US)

(72) Inventors: Rajat Mittal, Vienna, VA (US); Albert C. Lardo, Baldwin, MD (US); Jung Hee Seo, Baltimore, MD (US); Parastou Eslami, Baltimore, MD (US)

(21) Appl. No.: 13/868,665

(22) Filed: Apr. 23, 2013

Related U.S. Application Data
(60) Provisional application No. 61/652,385, filed on May 29, 2012.

(51) Int. Cl. A61B 6/03

(52) U.S. Cl. CPC A61B 6/032
USPC 600/426; 600/431

(57) ABSTRACT

An embodiment in accordance with the present invention provides a method for non-invasively determining the functional severity of coronary artery stenosis. The method includes gathering patient-specific data related to concentration of a contrast agent within a coronary artery of a patient using a coronary computed tomography angiography scan (CCTA). The patient-specific data is used to calculate a patient-specific transarterial attenuation gradient for the coronary artery of the patient. The patient specific transarterial attenuation gradient is compared to previously collected data to determine an estimate of a pressure gradient and/or fractional flow reserve (FFR) for the patient. As more data is collected, the data can be added to the database in order to increase the accuracy of future assessments. The database can also be enhanced by adding data generated by canonical models and mathematical analysis.

### CROSS REFERENCE TO RELATED APPLICATIONS

This application claims the benefit of U.S. Provisional Patent Application No. 61/652,385 filed on May 29, 2012, which is incorporated by reference, herein, in its entirety.

### FIELD OF THE INVENTION

The present invention relates generally to cardiology. More particularly, the present invention relates to a method for determining pressure gradients and fractional flow reserve.

### BACKGROUND OF THE INVENTION

The coronary arteries supply the myocardium, or muscle of the heart with oxygen and nutrients. Over time the coronary arteries can become clogged with cholesterol and other material known as plaque. Coronary artery disease results from this buildup of plaque within the walls of the coronary arteries. Excessive plaque build-up can lead to diminished blood flow through the coronary arteries and eventually chest pain, ischemia, and heart attack. Coronary artery disease can also weaken the heart muscle and contribute to heart failure, a condition where the heart cannot pump blood to the rest of the body, and arrhythmias, which are changes in the normal beating rhythm of the heart. Coronary artery disease is quite common, and, in fact, is the leading cause of death for both men and women in the United States.

There are several different diagnostics that are currently used to assess coronary artery disease and its severity. Non-invasive tests can include electrocardiograms, biomarker evaluations from blood tests, treadmill tests, echocardiography, single positron emission computed tomography (SPECT), and positron emission tomography (PET). Unfortunately, these non-invasive tests do not provide data related to the size of a coronary lesion or its effect on blood flow.

While CT scans and MRI can be used to visualize the size of the lesion, lesion size does not necessarily correlate to the functional significance of the lesion. Therefore, additional assessments have been developed to determine functional significance of coronary artery lesions. Generally, pressure gradient (PG) and fractional flow reserve (FFR) are the gold standard for assessments used to determine the functional significance of coronary artery stenosis. These metrics are currently determined using diagnostic cardiac catheterization, a procedure in which a catheter is inserted into a peripheral artery and threaded through the vasculature to the relevant areas of the coronary arteries. FFR is determined by calculating the ratio of the mean blood pressure downstream from a lesion divided by the mean blood pressure upstream from the same lesion. These pressures are measured by inserting a pressure wire into the patient during the diagnostic cardiac catheterization procedure. While this procedure provides an accurate measure of FFR for determining the functional severity of the coronary stenosis, it is only obtained after the risk and cost of an invasive procedure have already been assumed.

FFR can also be estimated based on a highly complex computational fluid dynamics modeling in CT derived, patient-specific coronary models. This approach requires a high level of sophistication, is computationally expensive, and requires that patient-specific data be transmitted out of the hospital environment to a third party vendor. It is expensive and can take several days to obtain results.

It would therefore be advantageous to provide a new method for determining the PG and/or FFR for a patient's coronary arteries using a non-invasive procedure with results that can be determined quickly and on-site.

### SUMMARY OF THE INVENTION

The foregoing needs are met, to a great extent, by the present invention, wherein in one aspect a method for determining a functional significance of coronary artery stenosis includes gathering patient-specific data related to concentration of a contrast agent within a coronary artery of a patient. The method also includes using the patient-specific data to calculate a patient-specific transarterial attenuation gradient for the coronary artery of the patient. The patient specific transarterial attenuation gradient is compared to data which has been generated or collected previously, to determine an estimate of a pressure gradient for the patient.

According to an aspect of the present invention, the method can be executed using a computer readable medium. A cardiac computed tomography scan is used to gather the patient specific data. A database of the previously collected data is compiled. The patient specific data and patient specific transarterial gradient can also be added to enhance the database. The database can be built using information chosen from at least one of the group of patient data, arterial model data, and analysis based data. A graphical view, tabular representation, or curve-fit equation of the previously collected data can be used for comparison to the patient-specific transarterial gradient. Constrictions of any geometric configuration in the coronary artery can be analyzed using the method. The patient-specific data can also be represented as a graph, table, or curve-fit equation of concentration of the contrast agent over a distance in the coronary artery.

In accordance with another aspect of the present invention, a system for determining a functional significance of coronary artery stenosis includes a computing device further including a computer readable medium. The computer readable medium is programmed for gathering patient-specific data related to concentration of a contrast agent within a coronary artery of a patient. The computer readable medium is also programmed for using the patient-specific data to calculate a patient-specific transarterial attenuation gradient for the coronary artery of the patient and comparing the patient specific transarterial attenuation gradient to previously collected data to determine an estimate of a pressure gradient for the patient.

In accordance with another aspect of the present invention, the patient-specific data is taken from computed tomography scan data. Therefore, the system can also include a computed tomography scanning device. The computed tomography scanning device is networked wirelessly or in a wired manner to the computing device. The computer readable medium can further be programmed for creating a database of the previously collected data. The patient specific data and patient specific transarterial gradient can be added to the database either manually or by the computer readable medium. The database can be built using information chosen from at least one of the group of patient data, arterial model data, and analysis based data and can be stored on the computing device. Further, the computer readable medium can be programmed for generating at least one of a graphical view, tabular representation, or curve-fit equation of the previously collected data for comparison to the patient specific transarterial gradient. Additionally, the patient-specific data is represented as at least one of a graph, a table, or a curve-fit equation of concentration of the contrast agent over a distance in the coronary artery.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a flow diagram of a method of determining the functional severity of coronary artery constriction, according to an embodiment of the invention.

FIGS. 2A-C illustrate schematic diagrams of a blood vessel having 25%, 50%, and 75% asymmetric constriction, respectively.

FIGS. 2D-F illustrate contrast graphs depicting spatio-temporal evolution of contrast in the asymmetrically constricted coronary arteries depicted in FIGS. 1A-C, according to an embodiment of the invention.

FIGS. 3A-C illustrate schematic diagrams of a blood vessel having 25%, 50%, and 75% symmetric constriction, respectively.

FIGS. 3D-F illustrate contrast graphs depicting spatio-temporal evolution of contrast in the symmetrically constricted coronary arteries depicted in FIGS. 1A-C, according to an embodiment of the invention.

FIGS. 4A and 4B illustrate a graph plotting contrast concentration at time=225 against position in the blood vessel for asymmetric and symmetric constrictions, respectively, according to an embodiment of the invention.

FIG. 5A illustrates a graph plotting percentage constriction against estimated values for transarterial attenuation gradient for both symmetric and asymmetric constrictions, according to an embodiment of the invention.

FIG. 5B illustrates a graph plotting pressure gradient against estimated values for transarterial attenuation gradient for both symmetric and asymmetric constrictions, according to an embodiment of the invention.

FIGS. 6A-6D illustrate schematic diagrams of simulated flow and contrast dispersion in a simple modeled artery with 75% stenosis using IB modeling, according to an embodiment of the invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a method for non-invasively determining the functional severity of coronary artery stenosis. The method includes gathering patient-specific data related to concentration of a contrast agent within a coronary artery of a patient using a computed tomography angiography scan (CTA). The patient-specific data is used to calculate a patient-specific transarterial attenuation gradient for the coronary artery of the patient. The patient specific transarterial attenuation gradient is compared to previously collected or generated data to determine an estimate of a pressure gradient and/or fractional flow reserve (FFR) for the patient. As more data is collected (or generated), the data can be added to the database in order to increase the accuracy of future assessments. The database can also be enhanced by adding data generated by canonical models and mathematical analysis.

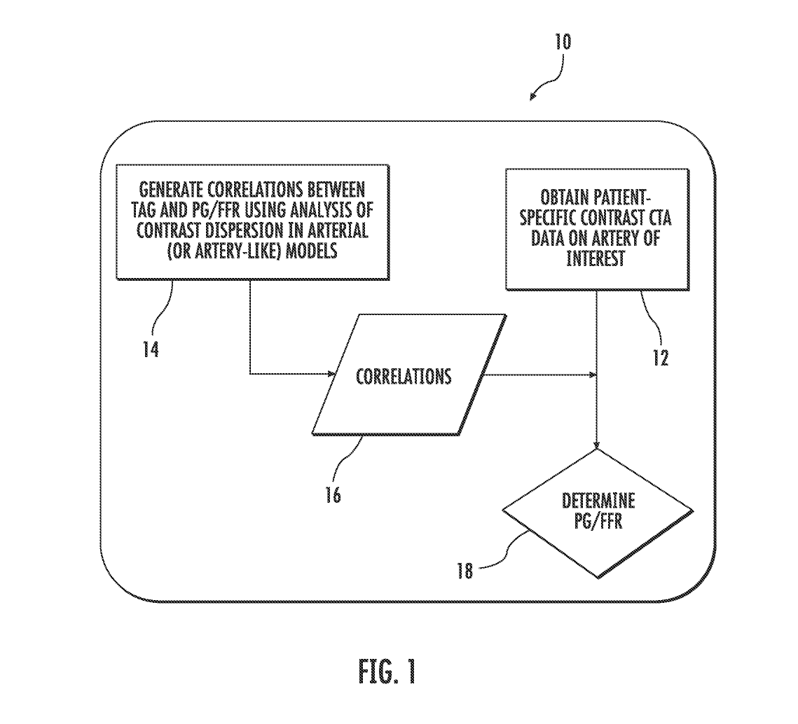

FIG. 1 illustrates a flow diagram of a method 10 of determining the functional severity of coronary artery constriction, according to an embodiment of the invention. The method includes a step 12 of obtaining patient specific contrast CTA data on an artery of interest. CTA scans are routinely acquired during angiography and myocardial perfusion scans. Therefore, in most cases the data can be obtained without requiring an additional procedure or scan. This allows physicians and diagnosticians to reduce invasive procedures for patients, as well as reduce the patients' exposure to radiation and contrast dye. Any suitable CT scanner known to one of skill in the art can be used. It is also expected that as medical imaging technology progresses, additional medical imaging devices currently in development or that will be developed in the future could also be used to execute the method.

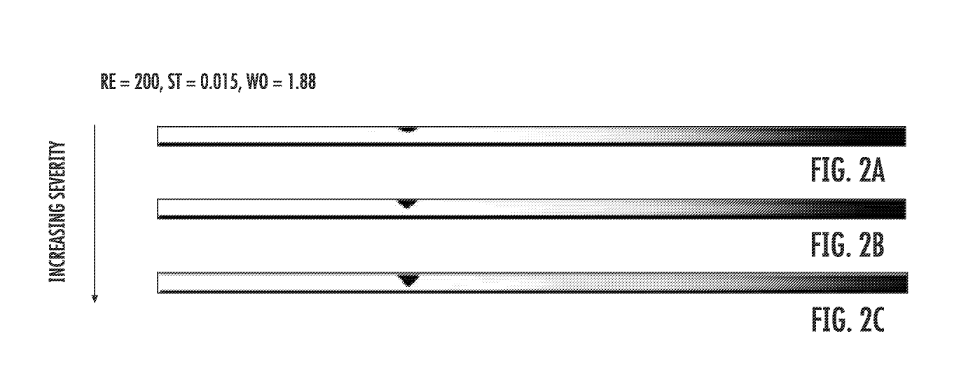

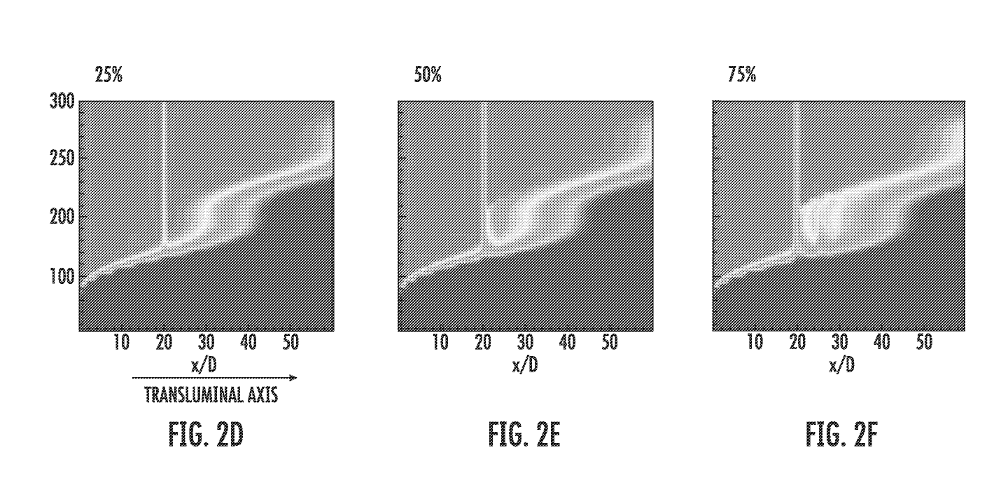

An example of patient specific data from the CTA scan that can be used in conjunction with the method described herein is data illustrating the dispersion of a contrast agent in a coronary artery over time. Other data known to one of skill in the art could, however, also be used to execute the method described herein. FIGS. 2A-F and 3A-F illustrate exemplary contrast CTA scan data that can be used in the execution of the method envisioned herein. FIGS. 2A-2C illustrate schematic diagrams of contrast dispersion in a blood vessel having 25%, 50%, and 75% asymmetric constriction, respectively. As illustrated in FIGS. 2A-2C, the contrast disperses farther along the blood vessel only having a 25% constriction than in the blood vessel having 50% or 75% constriction. The exemplary data in FIGS. 2A-2C is derived from a computer model having a Reynolds Number (Re) of 200, Strouhal number (St) of 0.015, and rotational velocity (W0) of 1.88. However, similar results would be obtained from a human patient.

FIGS. 2D-F further illustrate the dispersion of contrast in the coronary artery through contrast graphs depicting spatio-temporal evolution of contrast in the asymmetrically constricted coronary arteries depicted in FIGS. 2A-C. The graphs in FIGS. 2D-F illustrate the concentration of the contrast at different distances (x/D) along the coronary artery over time (t*) for coronary arteries having 25%, 50%, and 75% asymmetric constriction, respectively. The spike in each of the graphs at x/D=20 represents the stenosis.

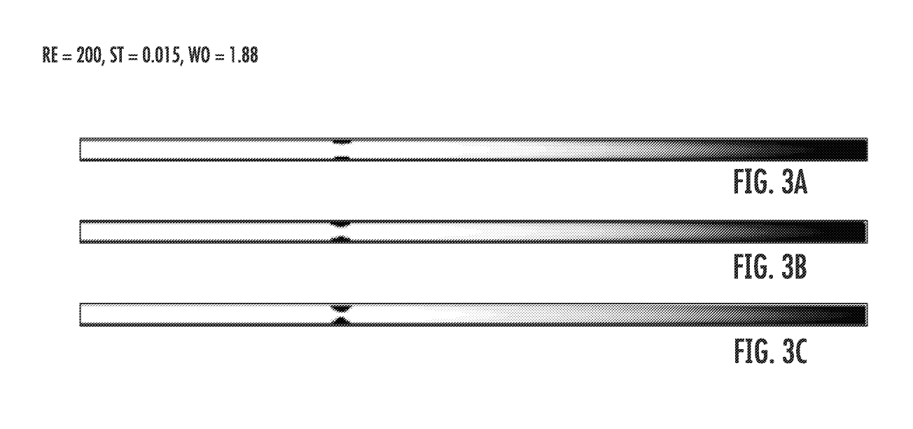

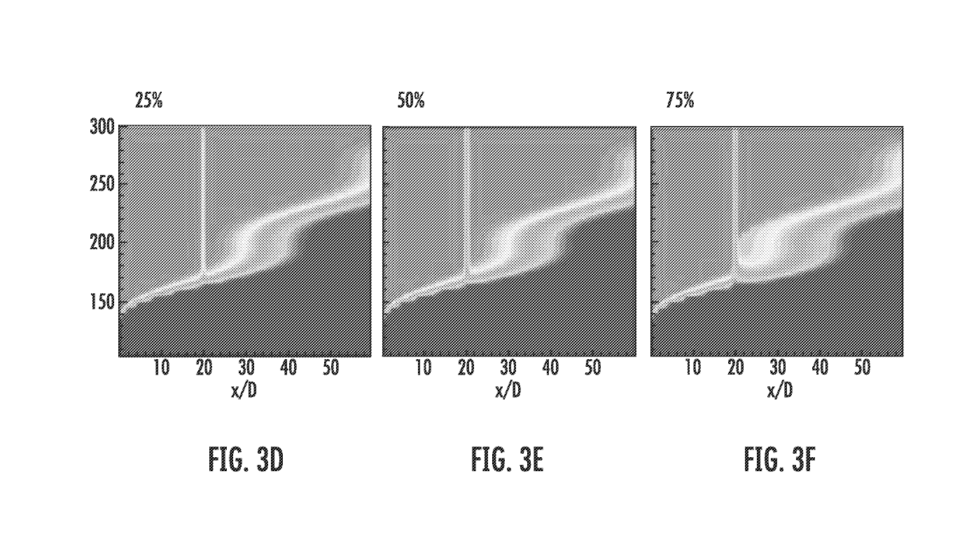

Similarly, FIGS. 3A-3C illustrate schematic diagrams of contrast dispersion in a blood vessel having 25%, 50%, and 75% symmetric constriction, respectively. As illustrated in FIGS. 3A-3C the contrast disperses farther along the blood vessel only having a 25% constriction than in the blood vessel having 50% or 75% constriction. The exemplary data in FIGS. 3A-3C is derived from a computer model having a Reynolds Number (Re) of 200, Strouhal number (St) of 0.015, and rotational velocity (W0) of 1.88. However, similar results would be obtained from a human patient.

FIGS. 3D-F further illustrate the dispersion of contrast in the coronary artery through contrast graphs depicting spatio-temporal evolution of contrast in the symmetrically constricted coronary arteries depicted in FIGS. 3A-C. The graphs in FIGS. 3D-F illustrate the concentration of the contrast at different distances (x/D) along the coronary artery over time (t*) for coronary arteries having 25%, 50%, and 75% symmetric constriction, respectively. The spike in each of the graphs at x/D=20 represents the stenosis.

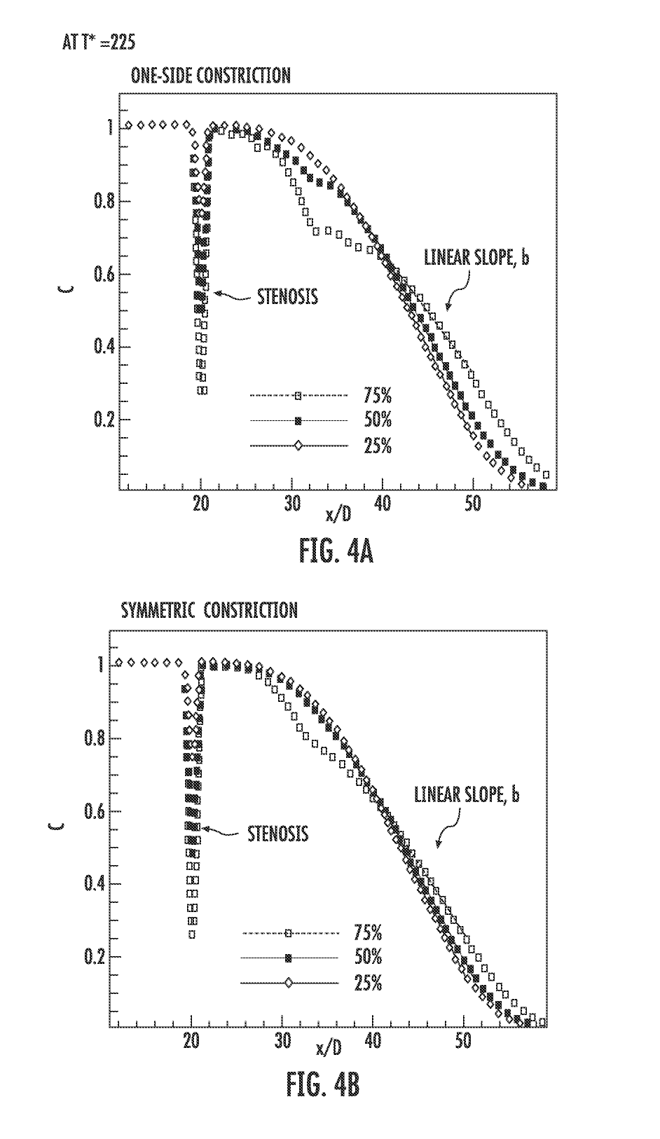

The patient-specific data is then processed to determine the transluminal attenuation gradient (TAG). The patient-specific data can be processed in any way known to one of skill in the art, such as by hand or using a computer readable medium programmed with the desired analysis method. As illustrated in FIGS. 4A and 4B, the data for the dispersion of contrast in vessels with both asymmetric and symmetric constrictions, is re-graphed, such that the concentration (C) at time 0=225 is plotted with respect to distance (x/D) along the coronary artery. Again, the stenosis can be seen in both FIGS. 4A and 4B at x/D=20. The linear slope (b) of the graphs for the asymmetric and symmetric constrictions at 25%, 50%, and 75% is determined, and is used to calculate TAG. The equation for TAG is as follows:

TAG=−b*100

For the exemplary data from the asymmetric and symmetric models, calculated TAG can be seen in Tables 1 and 2, below. TAG is then used to determine the PG and/or FFR for the coronary artery through comparison to a database of pre-existing information, which will be described in more detail below.

TABLE 1
Correlating Estimated TAG
with Stenotic Severity for Asymmetric Constriction

| Constriction | TAG |
|---|---|
| 25% | 4.990 |
| 50% | 4.696 |
| 60% | 4.384 |
| 75% | 3.317 |
| 80% | 2.786 |

TABLE 2
Correlating Estimated TAG with Stenotic Severity
for Symmetric Constriction

| Constriction | TAG |
|---|---|
| 25% | 5.019 |
| 50% | 4.740 |
| 60% | 4.532 |
| 75% | 3.848 |
| 80% | 3.055 |

Step 14 of the method includes generating correlations between TAG and PG/FFR using any or all of data from patient-specific testing, canonical models, and mathematical analysis. It should also be noted that any other means of building correlations and a database of these correlations known to one of skill in the art can be used, and the examples described herein should not be considered limiting. Using data from a number of sources will create a robust database that will allow the physician or diagnostician to make an accurate estimate of PG/FFR for the specific patient being tested. As more patients are tested, this patient-specific data can also be added to the database, with permission, in order to enhance the accuracy of the database. Mathematical variations on patient specific-data can also be included in the database. Canonical models, such as those used as examples for FIGS. 2A-F and 3A-F can also be generated and added to the database. The database can be held and maintained on a computer readable medium, fixed computer, computer server, or any other storage device known to one of skill in the art.

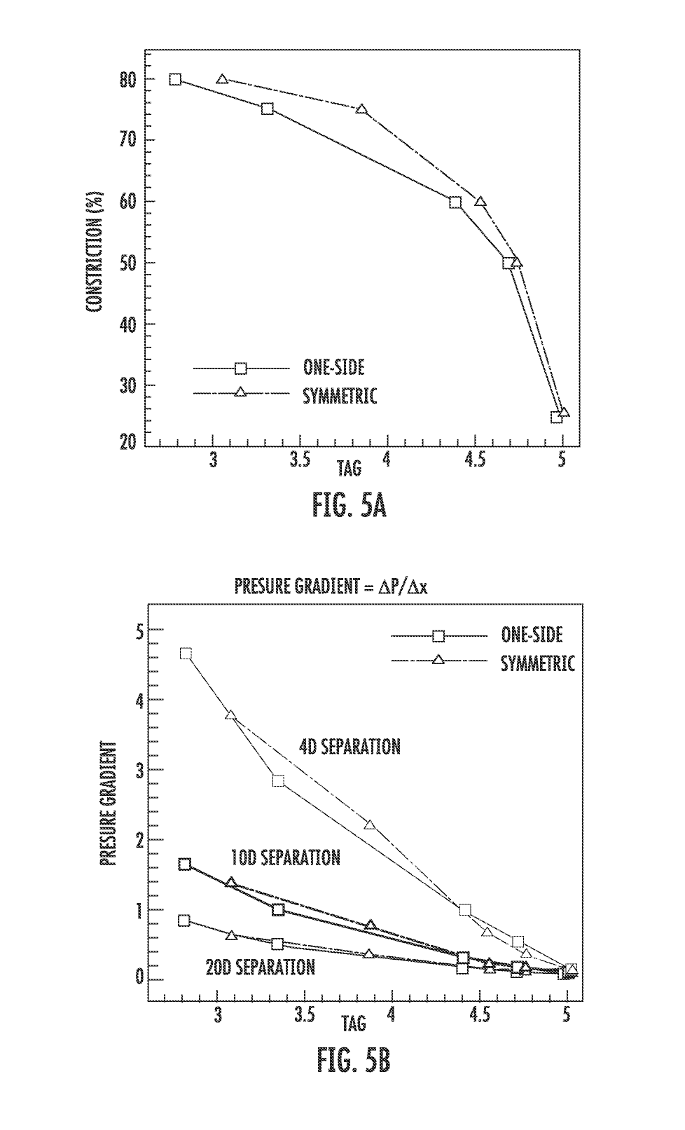

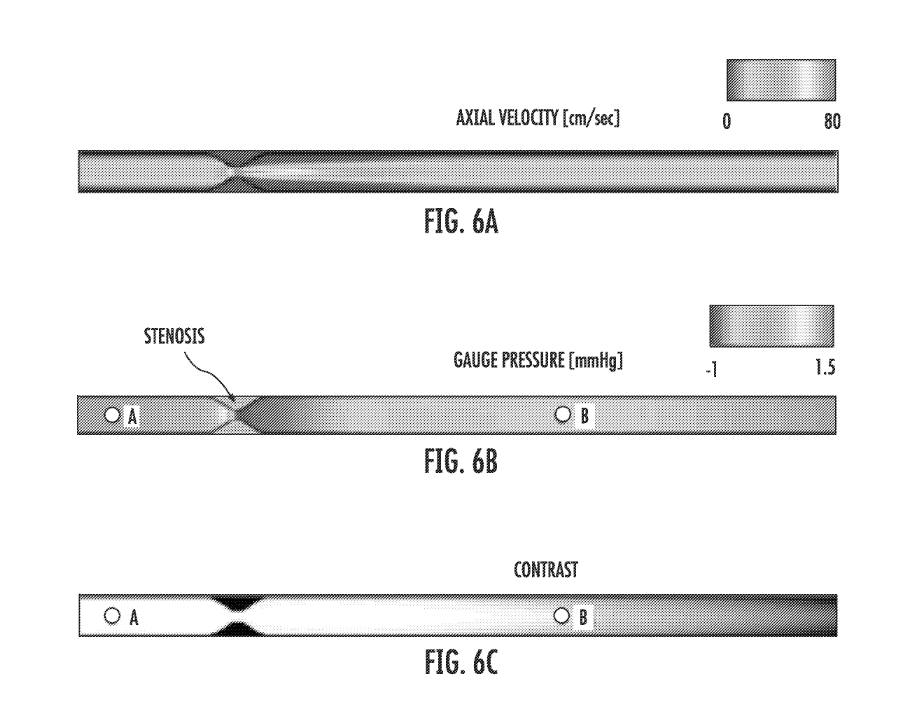

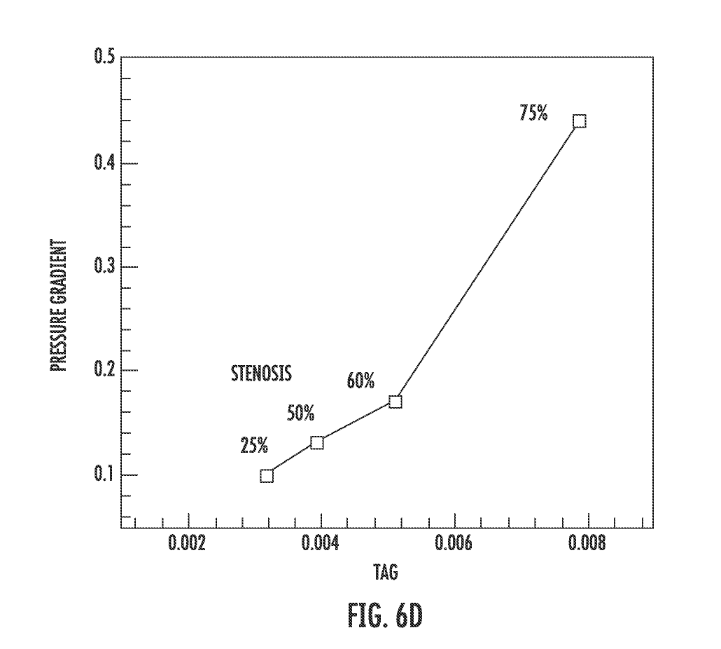

Correlations can then be made in step 16, using the data collected in step 14, as illustrated in FIG. 1. The correlation data can be generated and stored in a computer readable medium that is programmed to generate and store such correlations. Alternately, any other suitable means known to one of skill in the art could be used. The information stored in the database can be presented in a number of different ways including but not limited to spreadsheets, tables, or graphs. The information can also be represented as a curve-fit equation. Such correlations can be seen in Tables 3 and 4 below, which show the pressure gradient per constriction for both symmetric and asymmetric stenosis. FIGS. 5A and 5B also illustrate these correlations. FIG. 5A illustrates TAG plotted by percentage constriction, and FIG. 5B illustrates TAG plotted by pressure gradient. FIGS. 6A-D, also illustrate the correlation between TAG and pressure gradient, which can be used according to the present method. FIGS. 6A-C illustrate preliminary simulations of flow and contrast dispersion in a simple modeled artery with 75% stenosis using an IB method. FIG. 6D illustrates the correlation between non-dimensionalized trans-stenotic pressure and contrast gradients between points A and B illustrated in FIGS. 6A-C for stenosis severities ranging from 25% to 75%.

TABLE 3
Correlating Estimated TAG with Pressure Gradient
(Asymmetric Constriction)

| | Pressure Gradient (ΔP/Δx) | | |
|---|---|---|---|
| Constriction | Δx = 20D | 10D | 4D |
| 25% | 0.100 | 0.106 | 0.133 |
| 50% | 0.132 | 0.188 | 0.552 |
| 60% | 0.181 | 0.326 | 0.993 |
| 75% | 0.488 | 0.995 | 2.844 |
| 80% | 0.834 | 1.642 | 4.660 |

TABLE 4
Correlating Estimated TAG with Pressure Gradient
(Symmetric Constriction)

| | Pressure Gradient (ΔP/Δx) | | |
|---|---|---|---|
| Constriction | Δx = 20D | 10D | 4D |
| 25% | 0.099 | 0.104 | 0.127 |
| 50% | 0.120 | 0.154 | 0.361 |
| 60% | 0.149 | 0.232 | 0.670 |
| 75% | 0.349 | 0.749 | 2.198 |
| 80% | 0.628 | 1.373 | 3.779 |

In step 18, illustrated in FIG. 1, the patient-specific PG/FFR is determined by comparing the patient-specific TAG number to correlation information in the database. This step can be carried out in a number of different ways. For instance, the physician or diagnostician can input the patient's TAG number into a computer program for comparison to the database and generation of a PG/FFR number. If a computer system is not available, the physician or diagnostician can also visually compare the patient-specific TAG number to a graphical image of correlations between TAG numbers and PG/FFR, such as the one illustrated in FIG. 6D. Alternately, the physician or diagnostician can also use a table or a curve-fit equation to analyze the patient-specific data. The PG/FFR can then be used to assess the functional severity of the stenosis for that particular patient.

The proposed method is described herein with respect to assessment of the functional severity of constrictions in the coronary arteries. However, target anatomy need not be confined to the coronary arteries. This could be equally useful in performing assessments of other blood vessels, no matter the location. The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method for determining a functional significance of coronary artery stenosis comprising:
gathering patient-specific data related to concentration of a contrast agent within a coronary artery of a patient;
using the patient-specific data to calculate a patient-specific transarterial attenuation gradient for the coronary artery of the patient; and
comparing the patient specific transarterial attenuation gradient to previously collected data to determine an estimate of a pressure gradient for the patient.

2. The method of claim 1 further comprising executing the method using a computer readable medium.

3. The method of claim 1 further comprising performing a cardiac computed tomography scan to gather the patient specific data.

4. The method of claim 1 further comprising creating a database of the previously collected data.

5. The method of claim 4 further comprising adding the patient specific data and patient specific transarterial gradient to the database.

6. The method of claim 4 further comprising building the database using information chosen from at least one of the group of patient data, arterial model data, and analysis based data.

7. The method of claim 1 further comprising generating at least one of a graphical view, tabular representation, or curve-fit equation of the previously collected data for comparison to the patient specific transarterial gradient.

8. The method of claim 1 further comprising analyzing asymmetric constrictions.

9. The method of claim 1 further comprising analyzing symmetric constrictions.

10. The method of claim 1 wherein the patient-specific data is represented as at least one of a graph, a table, or a curve-fit equation of concentration of the contrast agent over a distance in the coronary artery.

11. A system for determining a functional significance of coronary artery stenosis comprising:
a computing device further comprising a computer readable medium programmed for:
gathering patient-specific data related to concentration of a contrast agent within a coronary artery of a patient;
using the patient-specific data to calculate a patient-specific transarterial attenuation gradient for the coronary artery of the patient; and
comparing the patient specific transarterial attenuation gradient to previously collected data to determine an estimate of a pressure gradient for the patient.

12. The system of claim 11 wherein the patient-specific data comprises computed tomography scan data.

13. The system of claim 11 further comprising a computed tomography scanning device.

14. The system of claim 13 wherein the computed tomography scanning device is networked wirelessly or in a wired manner to the computing device.

15. The system of claim 11 further comprising creating a database of the previously collected data.

16. The system of claim 15 further comprising adding the patient specific data and patient specific transarterial gradient to the database.

17. The system of claim 15 further comprising building the database using information chosen from at least one of the group of patient data, arterial model data, and analysis based data.

18. The system of claim 15 further comprising storing the database on the computing device.

19. The system of claim 11 further comprising generating at least one of a graphical view, tabular representation, or curve-fit equation of the previously collected data for comparison to the patient specific transarterial gradient.

20. The system of claim 11 wherein the patient-specific data is represented as at least one of a graph, a table, or a curve-fit equation of concentration of the contrast agent over a distance in the coronary artery.
